(19) United States
(12) Patent Application Publication
(10) Pub. No.: US 2002/0009421 A1
(43) Pub. Date: Jan. 24, 2002

(54) Therapy following skin injury from exposure to ultraviolet radiation

(76) Inventors: Karol J. Wilder, St. Louis, MO (US); Joseph R. Schuh, St. Louis, MO (US)

Correspondence Address: HARNESS, DICKEY, & PIERCE, P.L.C, 7700 BONHOMME, STE 400, ST. LOUIS, MO, 63105, US

(21) Appl. No.: 09/866,196

(22) Filed: May 25, 2001

Related U.S. Application Data
(60) Provisional application No. 60/208,798, filed on Jun. 1, 2000.

(51) Int. Cl. A61K 7/42; A61K 31/42; A01N 43/80

(52) U.S. Cl. 424/59; 514/378

(57) ABSTRACT

There is provided a method of relieving pain, fever and/or inflammation in a subject suffering sunburn or other skin injury resulting from exposure to UV radiation, the method comprising orally administering to the subject a therapeutically effective amount of a selective COX-2 inhibitory drug.

### CROSS REFERENCE TO RELATED APPLICATION

This application claims priority to U.S. application Ser. No. 60/208,798 filed Jun. 1, 2000.

### FIELD OF THE INVENTION

The present invention relates to a new therapeutic method of treating a subject suffering injury to the skin as a result of exposure to ultraviolet radiation (“UV injury”), for example sunburn. In particular, the present invention relates to oral administration of a selective cyclooxygenase-2 inhibitory drug and to manufacture of a medicament containing such a drug that is useful in treatment of a subject suffering UV injury to the skin.

### BACKGROUND OF THE INVENTION

Exposure of the skin to ultraviolet (UV) light can produce immediate as well as long-term effects. The predominant acute effects of exposure to UV light include sunburn and vitamin D synthesis. Chronic exposure to UV light can produce photodamaged skin which exhibits wrinkling blotchiness, telangiectasia and a roughened, weather-beaten appearance as well as the more serious consequence of the development of melanoma or nonmelanoma skin cancer. Although the risk of skin cancer does not correlate well with cumulative exposure to UV light, skin cancers are generally considered long-term sequela of exposure to UV light.

Sunburn can appear from one to 24 hours after exposure to UV light such as from the sun and can involve from mild symptoms such as erythema or redness with subsequent scaling to more severe symptoms such as pain, swelling or edema, tenderness and blistering. Systemic symptoms can also appear particularly if a large portion of the body surface is affected. Such symptoms can include fever, chills, weakness and in severe instances, shock. UV-B radiation in the wavelength of from about 290 to about 320 nm is believed to be responsible for the majority of photodamage to the skin produced by exposure to the sun.

The mechanism of photodamage to the skin is not fully understood. Nevertheless, exposure to UV-B radiation has been shown to produce a skin swelling as a result of increased vascular permeability and edema, neurtophil infiltration, increased prostaglandin levels and increased expression of the cyclooxygenase-2 (COX-2) gene (Wilgus et al., Prostaglandins & other Lipid Mediators 62:367-384:367-384, 2000). In addition, the topical application of the specific COX-2 inhibitor, celecoxib was reported to reduce many of the parameters of UV-B mediated injury (Id.). Nevertheless, this study reporting these results was limited to topical application of the drug and the researchers suggested that oral administration failed to block the cutaneous inflammatory response after exposure of skin to UV-B radiation. Another group studying the long-term development of tumors following exposure to UV radiation, reported that oral administration of the COX-2 inhibitor, celecoxib reduced tumor formation in animals chronically exposed to UV radiation (Fischer et al., Mol. Carcinog 25:231-240, 1999). In contrast to this, celecoxib failed to inhibit the acute UV-B mediated inflammatory response involving increased cell proliferation and edema following acute or chronic exposure to UV radiation (Id.). The drug, however, was reported to block the UV-B induced prostaglandin synthesis. Thus, although these references reported that topical application of the COX-2 inhibitor, celecoxib reduced the acute photodamage to the skin produced by UV-B radiation, oral administration of the drug reportedly did not.

The highly regulated COX-2 gene product catalyzes the production of prostaglandins whose actions include mediation of inflammation, pain and fever in many pathologic conditions (for review see Crofford et al., Arthritis Rheum. 43:4-13, 2000; Katori, Inflamm. Res. 49:367-392, 2000). As a result, specific COX-2 inhibitors have been developed and numerous compounds have been reported having therapeutically and/or prophylactically useful selective COX-2 inhibitory effects. Among such compounds are a large number of substituted pyrazolyl benzenesulfonamides as reported in U.S. Pat. No. 5,466,823 to Talley et al., including for example the compound 4-[5-(4-methylphenyl)-3-(trifluoromethyl)-1H-pyrazol-1-yl]benzenesulfonamide, also referred to herein as celecoxib (I), and the compound 4-[5-(3-fluoro-4-methoxyphenyl)-3-difluoromethyl)-1H-pyrazol-1-yl]benzenesulfonamide, also referred to herein as deracoxib (II).

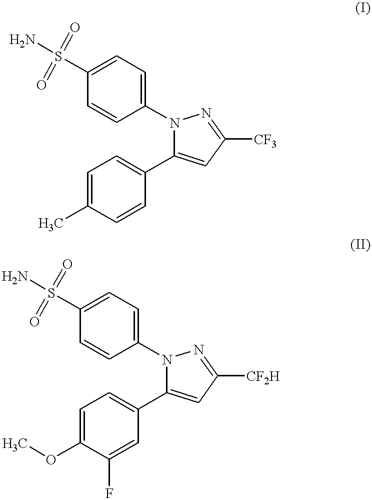

Other compounds reported to have therapeutically and/or prophylactically useful selective COX-2 inhibitory effect are substituted isoxazolyl benzenesulfonamides as reported in U.S. Pat. No. 5,633,272 to Talley et al., including the compound 4-[5-methyl-3-phenylisoxazol-4-yl]benzenesulfonamide, also referred to herein as valdecoxib (III).

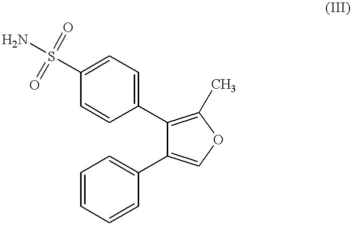

Still other compounds reported to have therapeutically and/or prophylactically useful selective COX-2 inhibitory effect are substituted (methylsulfonyl)phenyl furanones as reported in U.S. Pat. No. 5,474,995 to Ducharme et al., including the compound 3-phenyl-4-[4-(methylsulfonyl)phenyl]-5H-furan-2-one, also referred to herein as rofecoxib (IV).

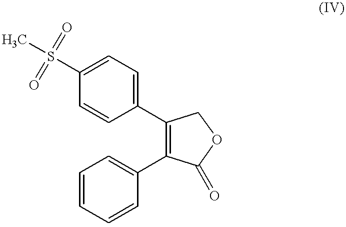

U.S. Pat. No. 5,981,576 to Belley et al. discloses a further series of (methylsulfonyl)phenyl furanones said to be useful as selective COX-2 inhibitory drugs, including 3-(1-cyclopropylmethoxy)-5,5-dimethyl-4-[4-(methylsulfonyl)phenyl]-5H-furan-2-one and 3-(1-cyclopropylethoxy)-5,5-dimethyl-4-[4-(methylsulfonyl)phenyl]-5H-furan-2-one.

U.S. Pat. No. 5,861,419 to Dube et al. discloses substituted pyridines said to be useful as selective COX-2 inhibitory drugs, including for example the compound 5-chloro-3-(4-methylsulfonyl)phenyl-2-(2-methyl-5-pyridinyl)pyridine, also referred to herein as etoricoxib (V).

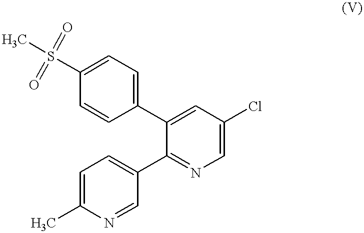

European Patent Application No. 0 863 134 discloses the compound 2-(3,5-difluorophenyl)-3-[4-(methylsulfonyl)phenyl]-2-cyclopenten-1-one said to be useful as a selective COX-2 inhibitory drug.

U.S. Pat. No. 6,034,256 to Carter et al. discloses a series of benzopyrans said to be useful as selective COX-2 inhibitory drugs, including the compound (S)-6,8-dichloro-2-(trifluoromethyl)-2H-1-benzopyran-3-carboxylic acid (VI).

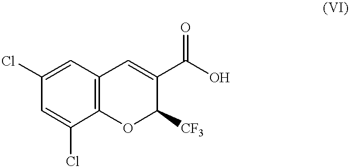

International Patent Publication No. WO 00/24719 discloses substituted pyridazinones said to be useful as selective COX-2 inhibitory drugs, including the compound 2-(3,4-difluorophenyl)-4-(3-hydroxy-3-methyl-1-butoxy)-5-[4-(methylsulfonyl)phenyl]-3-(2H)-pyridazinone.

Many patents relating to selective cyclooxygenase-2 inhibitory compounds, including those cited above, disclose utility of such compounds for relief of pain, fever and inflammation associated with a wide variety of conditions. However, hitherto there has been no disclosure or suggestion that oral administration of a selective cyclooxygenase-2 inhibitory drug might be useful in treating acute photodamage to the skin resulting from exposure to ultraviolet radiation, such as, for example sunburn.

International Patent Publication No. WO 99/13799 discloses co-administration of a selective cyclooxygenase-2 inhibitory drug with an opioid analgesic for relief of pain, and suggests that the dosage rate of the opioid analgesic can be reduced by such co-administration. International Patent Publication No. WO 99/21585 discloses a pharmaceutical composition comprising a selective cyclooxygenase-2 inhibitory drug and a second drug selected from acetaminophen and opiate compounds. These publications do not specifically identify utility in the context of UV injury, such as sunburn, of the skin.

A need exists for a therapeutic method of use of orally deliverable pharmaceutical compositions to provide effective relief of symptoms associated with UV injury to the skin including pain, fever and/or inflammation. A particular need exists for such a method giving such relief through selective inhibition of cyclooxygenase-2 (COX-2), without the undesirable side-effects associated with inhibition of cyclooxygenase-1 (COX-1) that can occur with conventional non-steroidal anti-inflammatory drugs (NSAIDs). An especial need exists for a method giving such relief while minimizing dosage rates of co-administered opioid drugs.

### SUMMARY OF THE INVENTION

It has now been discovered that a selective COX-2 inhibitory drug can provide a surprisingly effective and surprisingly rapid relief of pain and other manifestations of acute UV injury to skin, for example sunburn.

Accordingly, there is now provided a therapeutic method comprising orally administering to a mammalian, preferably human, subject suffering UV injury to skin a therapeutically effective amount of a selective COX-2 inhibitory drug. The symptoms of the UV injury are thus treated and, preferably, at least one of an analgesic, antipyretic and anti-inflammatory response is obtained.

In a particular embodiment, the method further comprises orally administering, in co-therapy with the selective COX-2 inhibitory drug, a second analgesic drug, preferably an opioid, at a dosage rate substantially lower than that normally administered for relief of pain when the second drug is used alone.

In another embodiment, the present invention is directed to a kit for treating or preventing UV injury to skin. The kit comprises a COX-2 inhibitory drug packaged with instructions for orally administering the drug to a mammalian subject, preferably a human subject, for treating or preventing UV injury to the skin. Preferably, the COX-2 inhibitor is present in an amount for orally administering the drug to produce at least one of an analgesic, antipyretic and anti-inflammatory response in the subject. In one aspect, this embodiment is directed to a packaged pharmaceutical comprising a COX-2 inhibitor prepared in dosage form for oral administration to treat skin injury from UV exposure. Such preparation is for the purpose of using the drug to treat or prevent skin injury from UV exposure and the preparation can comprise in whole or in part the preparation of a package label or package insert having instructions for administration to treat skin injury from UV exposure or an equivalent of such instructions.

What constitutes a therapeutically effective amount, or dose, of a selective COX-2 inhibitory drug depends, among other factors, on the particular drug being administered, the body weight of the subject and the severity of the photodamage to the skin and symptoms thereof including pain, fever or inflammation. Normally an effective celecoxib dose will be found in the range of about 1 to about 6 mg/kg body weight. For an average 75 kg subject, this range equates to a celecoxib dose of about 75 to about 450 mg. Proportionately smaller or larger doses can be appropriate for subjects having lesser or greater body weight. For selective COX-2 inhibitory drugs other than celecoxib, an appropriate dose is one that is therapeutically equivalent to the celecoxib doses indicated above. The therapeutically effective dose can be administered as needed, but typically administration 1 to about 4 times per day, in most cases 1 or 2 times a day, provides adequate continuing relief of pain, fever and/or inflammation resulting from UV injury to the skin.

By contrast with therapeutic methods involving NSAIDs lacking selectivity for inhibition of COX-2, highly effective relief of pain, fever and/or inflammation associated with UV injury can be obtained without the side-effects commonly associated with COX-1 inhibition. Thus the method of the present invention is suitable where NSAIDs are contraindicated, for example in patients with peptic ulcers, gastritis, regional enteritis, ulcerative colitis or diverticulitis, patients with a recurrent history of gastrointestinal lesions, patients with gastrointestinal bleeding, coagulation disorders including anemia such as hypothrombinemia, hemophilia and other bleeding problems, or kidney disease, patients prior to surgery, or patients taking anticoagulants.

Other features and advantages of the invention will be in part apparent and in part pointed out hereinafter.

### DETAILED DESCRIPTION OF THE INVENTION

The present invention provides a method of relieving the pathologic conditions associated with exposure of the skin to UV radiation, in particular, at least one of pain, fever and inflammation in a mammalian subject The method comprises orally administering to the subject a therapeutically effective amount of a selective COX-2 inhibitory drug.

The method of the invention is useful for treatment of non-human mammals, including domestic, farm and exotic animals, for example dogs, horses and zoo animals, but is primarily useful for treatment of human subjects.

By UV light it is meant, electromagnetic energy having a wavelength between about 10 and 400 nm. The ultraviolet spectrum is arbitrarily divided into three major segments, UV-A light at wavelengths from about 320 to about 400 nm, UV-B light having wavelengths from about 290 to about 320 nm and UV-C light having wavelengths from about 10 to 290 nm. The UV-B portion of the UV spectrum is predominantly responsible for producing the redness or erythema of sunburn whereas, the UV-A light is approximately a thousandfold less efficient in producing skin hyperemia or sunburn. UV-C light from the sun does not reach the earth, but is absorbed by stratospheric ozone.

The method of treatment in the present invention includes treatment of existing photodamage to the skin as well as prophylactic treatment to prevent photodamage to the skin and/or symptoms related thereto. The term “prevent” or “preventing” as used herein in the context of preventing photodamage to the skin is intended to include diminishing the severity of photodamage to the skin and/or symptoms related thereto. Thus, in such instances in which exposure of the skin of an individual is anticipated, the selective COX-2 inhibitor can be administered prior to the exposure to the UV radiation. Such prior administration can be, for example, from about 5 to 15 minutes prior to the exposure up to about 24 hours prior to the exposure, depending upon the pharmacokinetic profile of the particular dosage formulation administered. Thus, for a rapidly bioavailable formulation, administration could be from about 5 to about 15 minutes prior to exposure to UV radiation. For less rapidly bioavailable formulations, administration could be from about 1 hour to about 4 hours prior to exposure and for delayed or sustained release formulations, administration could be from about 6 hours to about 24 hours prior to exposure to UV radiation.

Treating or preventing UV injury to the skin is intended to included alleviating or diminishing aspects of the injury which are of an acute nature, whether primary or subsequent to the photodamage and/or alleviating symptoms associated with the injury. The effectiveness of the COX-2 inhibitory drug in treating UV-elicited photodamage to the skin can be measured by assessing of the degree of severity of symptoms associated with the photodamage. For sunburn, the severity and/or duration of erythema, the progression to scaling, the presence or degree of pain, edema, tenderness, and/or blistering can be assessed or any combination thereof. The assessment can involve patient scoring of severity of such symptoms or by any other method known in the art. In cases in which a large portion of the body surface is affected, the effectiveness of treatment of symptoms such as fever, chills, and weakness can be assessed. In a preferred approach, the severity of inflammation, pain and/or fever indicates the effectiveness of the COX-2 inhibitory drug.

Selective COX-2 inhibitory drugs useful in the method of the invention include, without limitation, compounds having the formula (VII):

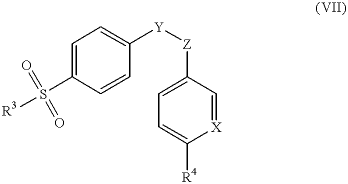

where R³ is a methyl or amino group, R⁴ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups. Preferred such five- to six-membered rings are cyclopentenone, furanone, methylpyrazole, isoxazole and pyridine rings substituted at no more than one position. Also useful are prodrugs that provide such selective COX-2 inhibitory compounds upon oral administration.

Illustratively, celecoxib, deracoxib, valdecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid and 2-(3,4-difluorophenyl)-4-(3-hydroxy-3-methyl-1-butoxy)-5-[4-(methylsulfonyl)phenyl]-3-(2H)-pyridazinone, more particularly valdecoxib and etoricoxib, and most particularly valdecoxib are useful in the method and composition of the invention.

The invention is illustrated herein with particular reference to celecoxib, and it will be understood that any other selective cyclooxygenase-2 inhibitory compound can, if desired, be substituted in whole or in part for celecoxib in the method herein described.

Selective COX-2 inhibitory drugs used in the method of the invention can be prepared by a process known per se, in the case of celecoxib, for example, by processes described in U.S. Pat. No. 5,466,863 to Talley et al. or in U.S. Patent No. 5,892,053 to Zhi & Newaz, both incorporated herein by reference. Other selective COX-2 inhibitory drugs can be prepared by processes known per se, including processes set forth in patent publications disclosing such drugs; for example in the case of valdecoxib in above-cited U.S. Pat. No. 5,633,272, and in the case of rofecoxib in above-cited U.S. Pat. No. 5,474,995.

A suitable dose of celecoxib, administered according to the method of the invention, is typically in the range of about 1 to about 6 mg/kg body weight, preferably about 1.3 to about 5.3 mg/kg body weight and more preferably about 2 to about 3.5 mg/kg body weight, for example about 2.7 mg/kg body weight. Depending on the body weight of the subject, a suitable dosage amount of celecoxib in an adult human is typically about 50 to about 400 mg, preferably about 75 to about 300 mg. Surprisingly good results can be obtained with dosage amounts less than 300 mg, for example about 100 mg or about 200 mg.

The doses set out above relate to a single administration, and can be repeated as needed. Generally no more than about 4 doses per day will be needed, and in most cases 1 or 2 doses per day will be found sufficient.

Celecoxib is highly hydrophobic; inclusion in the formulation of a wetting agent can provide wetting of celecoxib particles and can improve absorption in the gastrointestinal tract. Any suitable wetting agent can be used; presently preferred examples include polysorbate 80 and sodium lauryl sulfate.

Formulations useful in the present invention can be imbibable liquids or solid unit dosage forms. Celecoxib unit dosage forms useful in the invention typically contain about 10 mg to about 400 mg of celecoxib, for example, a 10, 20, 37.5, 50, 75, 100, 125, 150, 175, 200, 250, 300, 350, or 400 mg dose of celecoxib. Preferred unit dosage forms contain about 50 mg to about 400 mg of celecoxib. More preferred unit dosage forms contain about 100 mg to about 200 mg of celecoxib.

In an imbibable formulation, celecoxib can be present at any suitable concentration. Preferably the concentration is sufficiently high that the volume of liquid that has to be imbibed is not inconveniently great for the patient. For example, for a 200 mg dose, it is preferable that the concentration of celecoxib in an imbibable solution or suspension be not less than about 0.1%, so that the volume of solution or suspension to be imbibed is not greater than about 200 ml.

In a solid unit dosage form of celecoxib, the celecoxib is present at a minimum concentration of about 1%, preferably about 4%, more preferably about 10%, and still more preferably about 20%, by weight. The maximum concentration of celecoxib in a solid unit dosage form depends, among other factors, on the excipients present in the formulation, but is normally about 90%, preferably about 75% and more preferably about 50%, by weight.

The method of the present invention optionally further comprises oral administration, in co-therapy with the selective COX-2 inhibitory drug, of a second analgesic drug. In one embodiment the second analgesic drug can be another selective COX-2 inhibitor, but is preferably an opioid or other analgesic selected from narcotic analgesics, Mu receptor antagonists, Kappa receptor antagonists, non-narcotic (i.e., non-addictive) analgesics, monamine uptake inhibitors, adenosine regulating agents, cannabinoid derivatives, Substance P antagonists, neurokinin-1 receptor antagonists and sodium channel blockers. Preferred co-therapies comprise co-administration, with a selective COX-2 inhibitory drug, of one or more compounds selected from aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lornoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac (see The Merck Index, 12th Edition, Therapeutic Category and Biological Activity Index, ed. S. Budavari (1996), pp. Ther-2 to Ther-3 and Ther-12 (Analgesic (Dental), Analgesic (Narcotic), Analgesic (Non-narcotic), Anti-inflammatory (Nonsteroidal)).

Particularly preferred co-therapies comprise use of a selective COX-2 inhibitory compound with an opioid compound, more particularly where the opioid compound is codeine, meperidine, morphine or a derivative thereof. Co-therapy permits reduction in the dosage of the opioid drug, with concomitant reduction or avoidance of undesirable side-effects of the opioid.

The compound to be administered in combination with the selective COX-2 inhibitory drug can be formulated separately therefrom or co-formulated with the selective COX-2 inhibitory drug in a single composition. Either or both of the drugs can be formulated in immediate-release, rapid-onset, sustained-release or dual-release form.

### EXAMPLE

The following Example is provided for illustrative purposes only and is not to be interpreted as limiting the scope of the present invention. The Example will permit better understanding of the invention and better perception of its advantages.

In Vivo Evaluation

1. Subject: An adult, Caucasian woman of light complexion whose skin had not been substantially exposed to strong sunlight for approximately six months.

2. Protocol: The subject was exposed to intense sunlight at a swimming pool for approximately six hours.

3. Therapeutic Regimen and Results: The subject awoke the next morning experiencing extreme pain and exhibiting redness and tenderness of the skin where it had been previously exposed to the sun. At that time the subject self-administered an oral dose of acetaminophen (Extra Strength Tylenol®) to relieve the pain. The subject experienced no relief in pain during the next several hours. Later in the evening the subject was unable to sleep due to the continued pain. At that time the subject self-administered a 100 mg dose of Celebrex® (celecoxib). Within 1 hour following administration of the celecoxib the subject reported almost complete relief of pain and was able to sleep. Approximately twelve hours later the subject took a second dose of celecoxib to relieve the onset of discomfort due to the sunburn. In addition to pain relief the subject reported a rapid healing of the burn with no blistering or peeling of surface skin, as would have been expected from such a severe case of sunburn.

All references cited in this specification are hereby incorporated by reference. The discussion of the references herein is intended merely to summarize the assertions made by their authors and no admission is made that any reference constitutes prior art relevant to patentability. Applicants reserve the right to challenge the accuracy and pertinency of the cited references.

What is claimed is:

1. A method for treating or preventing UV injury to skin in a mammalian subject in need thereof, the method comprising orally administering to the subject a selective COX-2 inhibitory drug in an amount effective in treating or preventing the UV injury.

2. The method of claim 1 wherein orally administering the selective COX-2 inhibitory drug produces at least one of an analgesic, antipyretic and anti-inflammatory response.

3. The method of claim 2 wherein the mammalian subject is a human subject.

4. The method of claim 3 wherein the selective COX-2 inhibitory drug is a compound having the formula: 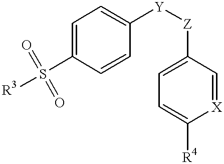
where R³ is a methyl or amino group, R⁴ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups; or a prodrug of such a compound.

5. The method of claim 4 wherein the five- to six-membered ring is selected from cyclopentenone, furanone, methylpyrazole, isoxazole and pyridine rings substituted at no more than one position.

6. The method of claim 3 wherein the selective COX-2 inhibitory drug is selected from celecoxib, deracoxib, valdecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid and 2-(3,4-difluorophenyl)-4-(3-hydroxy-3-methyl-1-butoxy)-5-[4-(methylsulfonyl)phenyl]-3-(2H)-pyridazinone and prodrugs thereof.

7. The method of claim 6 wherein the selective COX-2 inhibitory drug is selected from valdecoxib, etoricoxib and prodrugs thereof.

8. The method of claim 3 wherein the selective COX-2 inhibitory drug is administered in an amount therapeutically equivalent to about 50 to about 400 mg celecoxib.

9. The method of claim 3 wherein administering the selective COX-2 inhibitory drug is within about 24 hours after exposure to UV radiation.

10. The method of claim 3 wherein administering the selective COX-2 inhibitory drug is within about 4 hours prior to exposure to UV radiation.

11. The method of claim 3 that further comprises oral administration, in co-therapy with the selective COX-2 inhibitory drug, a second analgesic drug.

12. The method of claim 11 wherein the second analgesic drug is an opioid drug.

13. The method of claim 12 wherein the opioid drug is administered at a dosage substantially lower than that normally used for relief of pain when the opioid drug is used alone.

14. A kit for treating or preventing UV injury to skin, the kit comprising a COX-2 inhibitory drug packaged with instructions for orally administering the drug to a mammalian subject for treating or preventing UV injury to the skin.

15. The kit of claim 12 wherein the COX-2 inhibitory drug is in a formulation and amount for orally administering the drug to produce at least one of an analgesic, antipyretic and anti-inflammatory response in the subject.

16. The kit of claim 15 wherein the mammalian subject is a human subject.

17. The kit of claim 16 wherein the COX-2 inhibitory drug is a compound having the formula: 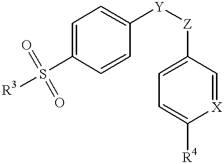
where R³ is a methyl or amino group, R⁴ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups; or a prodrug of such a compound.

18. The kit of claim 17 wherein the five- to six-membered ring is selected from cyclopentenone, furanone, methylpyrazole, isoxazole and pyridine rings substituted at no more than one position.

19. The kit of claim 16 wherein the selective COX-2 inhibitory drug is selected from celecoxib, deracoxib, valdecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid and 2-(3,4-difluorophenyl)-4-(3-hydroxy-3-methyl-1-butoxy)-5-[4-(methylsulfonyl)phenyl]-3-(2H)-pyridazinoneand prodrugs thereof.

20. The kit of claim 19 wherein the selective COX-2 inhibitory drug is selected from valdecoxib, etoricoxib and prodrugs thereof.

21. A package pharmaceutical comprising a COX-2 inhibitory drug prepared in dosage form for oral administration to treat skin injury from UV exposure.

22. The packaged pharmaceutical of claim 21 wherein the COX-2 inhibitory drug is in a formulation and amount for orally administering the drug to produce at least one of an analgesic, antipyretic and anti-inflammatory response in the subject.

23. The packaged pharmaceutical of claim 22 wherein the mammalian subject is a human subject.

24. The packaged pharmaceutical of claim 23 wherein the COX-2 inhibitory drug is a compound having the formula: 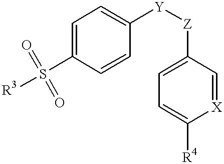
where R³ is a methyl or amino group, R⁴ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups; or a prodrug of such a compound.

25. The packaged pharmaceutical of claim 24 wherein the five- to six-membered ring is selected from cyclopentenone, furanone, methylpyrazole, isoxazole and pyridine rings substituted at no more than one position.

26. The packaged pharmaceutical of claim 23 wherein the selective COX-2 inhibitory drug is selected from celecoxib, deracoxib, valdecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid and 2-(3,4-difluorophenyl)-4-(3-hydroxy-3-methyl-1-butoxy)-5-[4-(methylsulfonyl)phenyl]-3-(2H)-pyridazinoneand prodrugs thereof.

27. The method of claim 26 wherein the selective COX-2 inhibitory drug is selected from valdecoxib, etoricoxib and prodrugs thereof.
